(19) United States
(12) Patent Application Publication
(10) Pub. No.: US 2009/0030368 A1
(43) Pub. Date: Jan. 29, 2009

(54) Device for Treatment of Breast Conditions

(75) Inventor: Brian H. Silver, Cary, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: MEDELA HOLDING AG, Baar (CH)

(21) Appl. No.: 12/178,432

(22) Filed: Jul. 23, 2008

Related U.S. Application Data
(60) Provisional application No. 60/961,830, filed on Jul. 24, 2007.

(51) Int. Cl. A61M 1/06

(52) U.S. Cl. 604/74

(57) ABSTRACT

A device for treatment of engorgement or edema of the breast has an orifice within which a breast nipple is received, a force-applying surface that presses against the adjacent breast to push fluid away from the nipple, and a part to which a motive force is applied to press the force-applying surface against the breast. The device can be used manually, worn within a bra, or used with a breastshield of a breastpump.

### REFERENCE TO RELATED APPLICATIONS

This application claims priority to U.S. Provisional Application Ser. No. 60/961,830 filed Jul. 24, 2007, the contents of which is incorporated by reference herein in its entirety.

### BACKGROUND OF THE INVENTION

1. Field of the Application

The invention relates generally to the treatment of breast conditions associated with childbirth, and particularly a device for the treatment of engorgement or edema in a mother's breast.

2. Description of the Related Art

Shortly after childbirth, mothers experience breast engorgement, a swelling of the mammary glands brought about by the presence of too much breast milk. When engorged, breasts grow larger and heavier with milk and may cause varying amounts of pain to the mother. New mothers may also experience edema of the breast, a condition in which an increase of fluids (for example, intravenous (IV) fluids or drugs such as pitocin administered during the childbirthing process) are accumulated in the breast tissue and cause swelling of the breast. Such fluids may take time (e.g., seven to fourteen days) after childbirth to diminish.

Mothers who become engorged or are edemic often have difficulty nursing, this difficulty extending to breastpumping. A technique referred to as “reverse pressure softening” has been used in the past to help alleviate these conditions. Reverse pressure softening softens the areola of the breast, thereby making latching (initial suckling attachment by the baby) and removal of the milk from the mother easier. This technique moves some of the swelling, resulting from the milk and other fluids, backward and upward into the breast to soften the areola so that it can change shape and extend the nipple.

Reverse pressure softening involves a physical manipulation of the breast to push fluid back away from the nipple. Specifically, a mother positions her fingers on either side of the nipple, and pushes gently but firmly inwardly towards her chest wall. This pressure is applied for a period of time such as one to three minutes, and may be repeated more than once if desired until the areola is softened.

This invention came about to provide a mother with a device that serves to apply pressure to the area surrounding the nipple in a manner that tends to produce a similar or improved result to the reverse pressure softening technique, so as to alleviate at least some of the effects of engorgement and edema.

### SUMMARY OF THE INVENTION

The present invention is a device that applies pressure to the area surrounding the nipple in a manner that tends to produce a similar or improved result to the reverse pressure softening technique, so as to alleviate at least some of the effects of engorgement and edema.

In one embodiment, the device has a diaphragm-like element that on one side is provided with an opening surrounded by a raised circular hummock-like area and on the other side is provided with a reinforcing bead or similar supporting structure to provided some rigidity. This embodiment can be adapted to fit on a handle or other support structure, or over the receiving end of a standard breastshield of a breastpump.

In another embodiment, the device is a ring constructed of a soft resilient material and has an opening in the form of a small channel in the center of the ring. This embodiment can be placed within a bra.

In yet another embodiment, the device is a solid toroidal ring with a raised area that extends into a well in the center of the ring. The well receives the nipple and adjacent breast. This embodiment also can be worn within a bra.

In still another embodiment, the device is an insert adapted to fit within the breastshield of a breastpump. The insert matches the contour of the breastshield and has a toroidal portion that massages the breast once the insert is pressed against the breast.

In a further embodiment, the device is a diaphragm-like element with a hummock-like raised area extending into a more pronounced elongate nipple tunnel. The embodiment has a circumferential portion that is designed to fit over the rim of a breastshield.

In yet a further embodiment, the device is a foam ring with a nipple receiving well in the center of the ring, the device attached to a handle. The interior of the nipple receiving well is closed at the bottom.

In still a further embodiment, the device has three parts: a rigid shell; a flexible base that mounts to the shell; and the engorgement/edema adapter element. The adapter has a toroidal-like ring part with a contour (toward the breast in use), which yields a hummock-like raised portion. The central region of the ring part slopes to an opening through which the nipple extends in use.

These as well as other aspects and advantages of the invention will become further apparent to those of skill in the art by reading the following detailed description, with reference where appropriate to the accompanying drawings. Further, it should be understood that the embodiments described herein are intended to illustrate the invention by way of example only.

### BRIEF DESCRIPTION OF THE DRAWINGS

Exemplary embodiments of the invention are described herein with reference to the drawings, in which:

FIG. 1 is a cross-sectional view of a first embodiment made in accordance with the present invention;

FIG. 2 is a cross-sectional view of a second embodiment made in accordance with the present invention;

FIG. 3 is a perspective view of a third embodiment made in accordance with the present invention;

FIG. 4 is a side view of a fourth embodiment made in accordance with the present invention;

FIG. 5 is a cross-sectional view of a fifth embodiment made in accordance with the present invention;

FIG. 6 is a partially cross-sectional view of a sixth embodiment made in accordance with the present invention;

FIG. 7 is a perspective view of a seventh embodiment made in accordance with the present invention;

FIG. 8 is a perspective view of the adapter shown in the embodiment of FIG. 7;

FIG. 9 is an exploded view of the embodiment of FIG. 7;

FIG. 10 is a perspective view of the flexible base attached to the shell and the adapter shown in the embodiment of FIG. 7;

FIG. 11 is an embodiment similar to that of FIG. 1 shown mounted with a handle;

FIG. 12 is a perspective view of the eighth embodiment made in accordance with the present invention; and

FIG. 13 is a side view of the adapter of the embodiment of FIG. 12.

### DETAILED DESCRIPTION OF EMBODIMENTS OF THE INVENTION

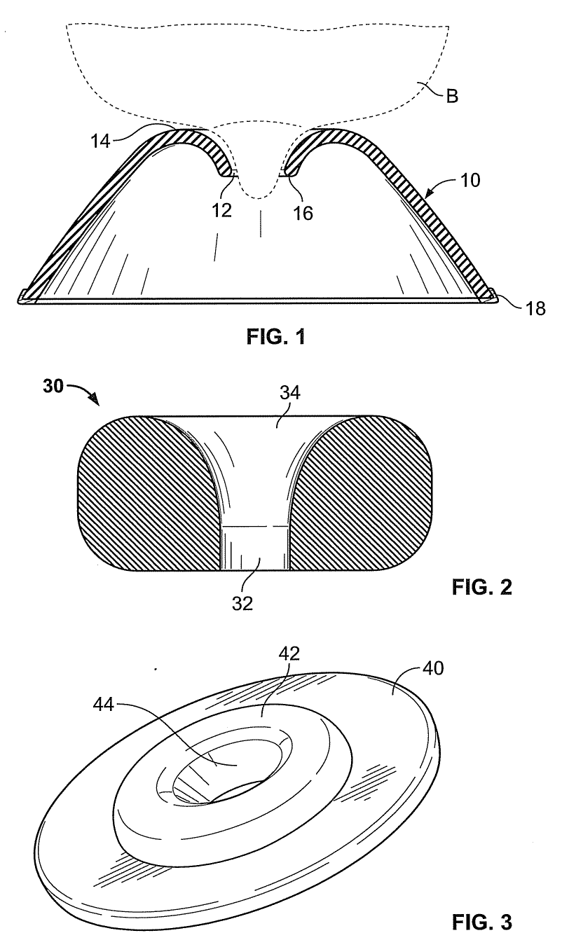

FIG. 1 shows a first embodiment which has a diaphragm-like element 10 which on one side presents a opening or orifice 12 surrounded by a raised circular hummock-like area 14. The opening 12 may be further defined by a bead 16 or similar supporting structure to give some rigidity surrounding its circumference. The opening 12 is sized to allow a nipple to fit therethrough, with the raised area 14 then close to the nipple and engaging the adjacent breast.

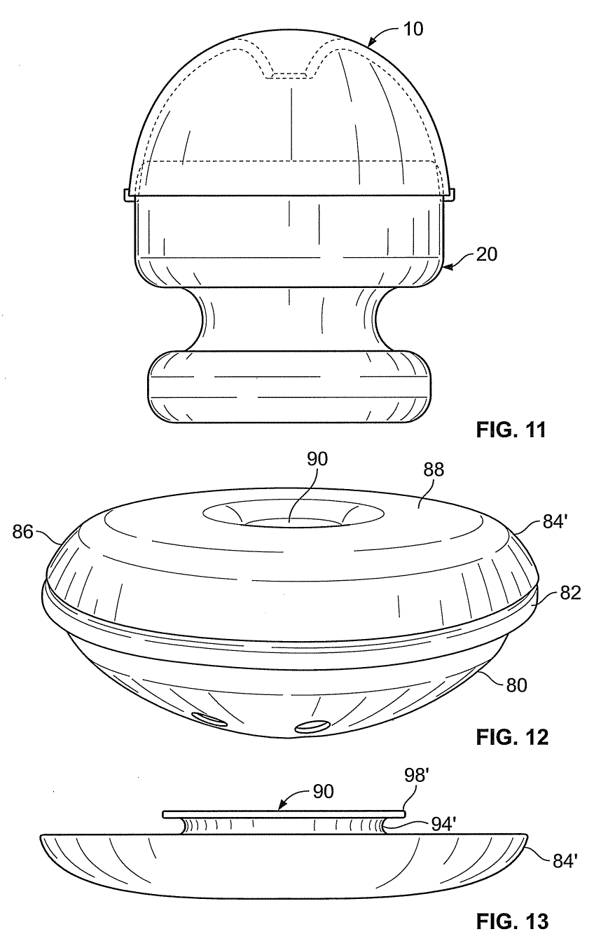

The opposed side of the element 10 is shown open here, and also may include a reinforcing bead 18, ring, or similar supporting structure, to give some rigidity about its circumference. The bead 18 may be adapted for mounting on a handle 20, as shown in FIG. 11. The element 10 is made of a flexible, resilient material, such as a silicone, with a suitable hardness or wall thickness to give it sufficient rigidity to push against the breast, yet permit it to stretch or deform when pressed toward the breast in use. This makes a kind of “rolling diaphragm” structure. For example, element 10 may have a wall thickness of at least 2 mm. Of course, a wall thickness of greater than 2 mm would also be suitable, provided that the element had sufficient rigidity and ability to stretch or deform.

That is, in use, the element 10 is positioned over the breast B with the nipple in opening 12. As element 10 is then pressed against the breast (using the bead 18 held by the palm of a hand, or with a handle 20 as shown in FIG. 11), pressure is first applied at the point of breast contact depicted in FIG. 1. The element 10 then begins to conform to the breast B upon being further pressed toward the breast, thus pushing against the breast in a manner starting at the nipple then extending radially outwardly from the nipple. In this way, fluid contained in the breast is moved away from the nipple.

As noted above with respect to FIG. 11, the element 10 could be adapted to fit on a handle 20 or other support or handling structure. Alternately, the element 10 could be adapted to fit over the shield (hood or funnel) end of a standard breastshield of a breastpump (not shown here, but see the discussion with respect to FIG. 5). That is, the embodiment of FIG. 1 can be adapted to snap-fit around the rim of the breastshield. The negative pressure (vacuum) of the breastpump could then be used to augment the effect of the element 10 (by pulling on the nipple/adjacent breast). The element 10 could be used manually (hand manipulated to press against the breast), or could be attached to a motor driven mechanism (such as the breastpump assembly vacuum pump) that would gently reciprocate the element 10 on a suitable mount.

FIG. 2 is another embodiment somewhat doughnut-like in shape. This version has a solid construction using a soft resilient material, such as foam, that is formed into the ring 30, as by molding the ring. An opening in the form of a small channel 32 is in the center of the ring 30. The contour and size of the interior sidewall 34 is designed to receive the nipple and adjacent breast tissue therein. When the ring 30 is pressed against the breast, the foam will compress against the breast, providing the radially outwardly or inwardly traveling pressure described above, which is desired. The foam material could be of multiple durometers in the same ring 30, to tailor the amount of massage as the ring 30 is pressed against the breast.

This FIG. 2 design could be placed within a bra, for example, and the mother could then gently press upon it with one hand while the ring 30 is held in place by the garment; or the bra can exert all the pressure. Thus, this embodiment could be supported in place handsfree (i.e., without the use of the mother's hands). Alternately, the ring 30 could be held on the mother's breast by a strap arrangement, again being held in place handsfree.

While designed for a solid foam structure in this embodiment of FIG. 2, it could just as well be semi-solid, or an inflated tube, or a viscous gel-filled interior, just to name a few other variations.

The embodiment of FIG. 3 is another doughnut-like construct. Here, a solid toroidal ring 40 is made of a flexible material, such as silicone, having some rigidity. A hummock 42 yields a raised area much like that of area 14 of the FIG. 1 embodiment. The raised area 42 then extends into a well 44 in the center of the ring 40 that receives the nipple and adjacent breast. This version of the invention can likewise be worn within a bra or similar garment.

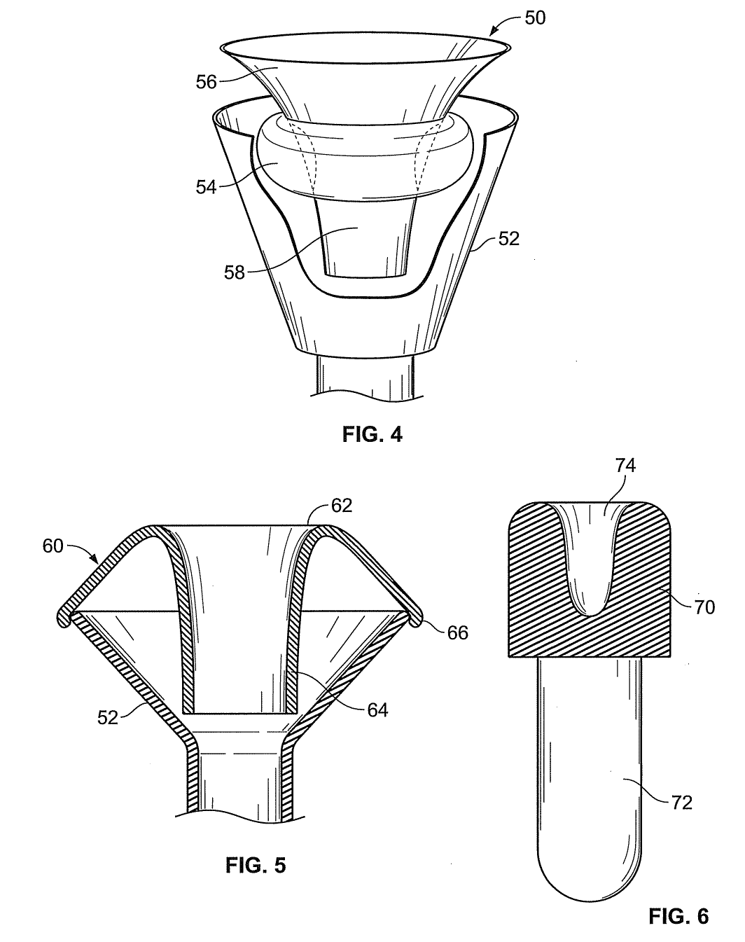

FIG. 4 shows an embodiment adapted for use with a conventional breastshield of a breastpump. Insert 50 is adapted to fit within the breastshield 52, in the same manner as other such inserts (typically used for sizing the breastshield for non-standard nipples/breasts). The insert 50 itself has a design generally matching the contour of the breastshield into which it will be inserted, and would be made of a flexible plastic, as will become evident hereafter. This insert has a toroidal portion 54 that is between the upper and lower parts 56, 58 of the insert. The toroidal portion is flexible and compressible in nature; it could be an inflated part, or made of an appropriate low durometer silicone in solid form. However made, it is designed to press against the sides of the insert 50 to press the latter against the breast tissue adjacent the nipple (received in the insert and extending past the toroidal portion 54). For instance, as the insert in the breastshield is pressed against the breast, the outboard side of the toroid will be pressed against the interior sidewall of the breastshield 52. This will in turn cause the inboard side of the toroid to press the sidewall of the insert, massaging the breast in the manner desired. The vacuum from the breastpump can be used to facilitate this massage, as it draws both the breast as well as the insert into the breastshield (since the toroidal portion 54 acts as a sliding gasket against the sidewall of the breastshield). If desired, the insert may be made fixed so as not to move as a result of the vacuum of the breast pump.

Note that if a hollow toroidal portion is used, it is quite possible to adapt it to be inflated/deflated using a pressure source (such as a tube connecting to a port on the toroidal portion). Toroidal portions could then furthermore be “stacked,” to inflate seriatim in a radially outwardly extending pattern.

FIG. 5 is another embodiment adapted for use with a conventional breastshield 52. In this variation, an element 60 reminiscent of the diaphragm-like element of FIG. 1 has a similar hummock-like raised area 62, which extends into a more pronounced elongate nipple tunnel 64. The element 60 has a circumferential portion 66 that is designed to fit over the rim of the breastshield 52. So arranged, pressing the breastshield 52 against the breast yields the same rolling diaphragm type action previously described. The downstream end of the nipple tunnel 64 can be designed to act as a stop of sorts to enhance the action. The element 60 could be made with more rigidity, however, yielding an action that is more of a pressing point force on the breast, rather than an expanding pressure zone.

It will be noted that where a breastshield adapted version of the invention is employed, the breastpump may be also adapted to operate in an engorgement/edema-reduction sequence or cycle. Such a cycle would be one in which the pressure source serves to move the inventive engorgement/edema reduction element and/or breast in a manner to apply the desired force on the breast.

FIG. 6 is a version similar to that of the foamaceous embodiment of FIG. 2. Here, the foam ring 70 is attached to a handle 72. The interior of the nipple receiving well 74 is closed at the bottom. The handle 72 is used to press the foam ring 70 against the breast in the desired manner.

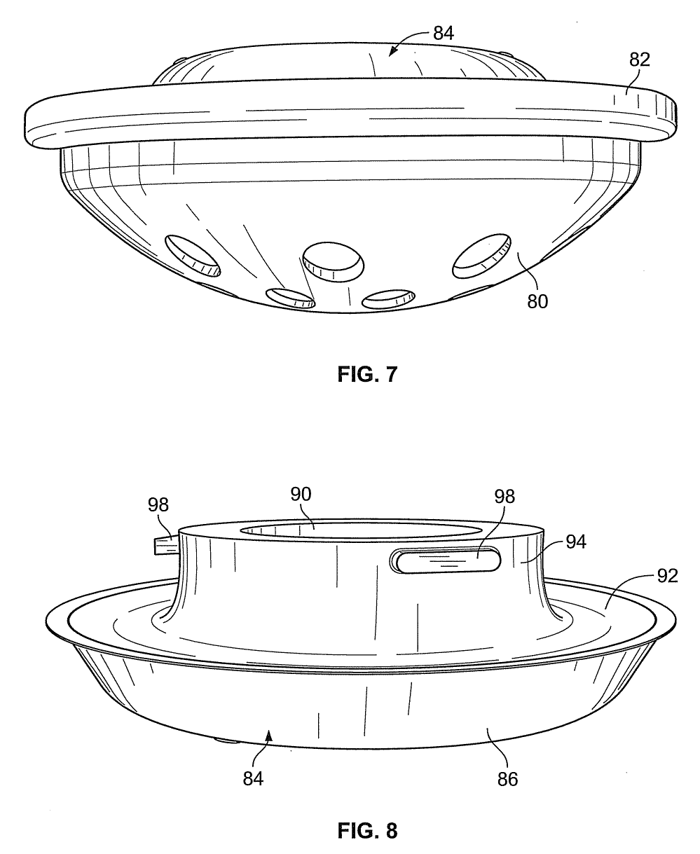

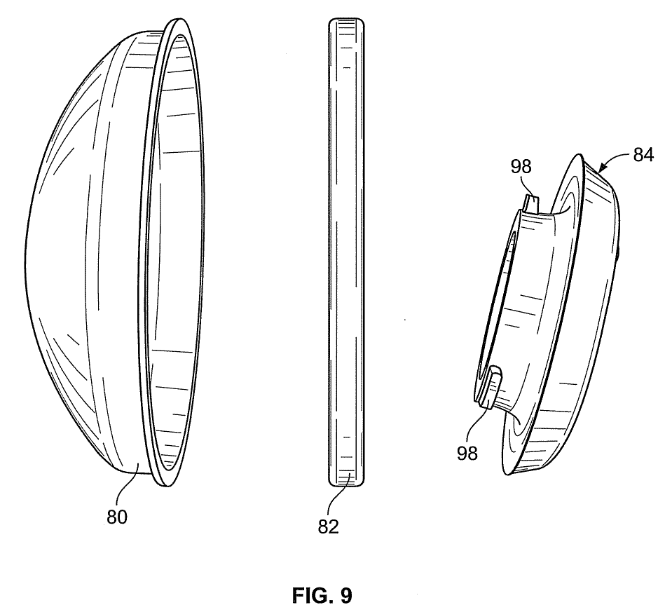

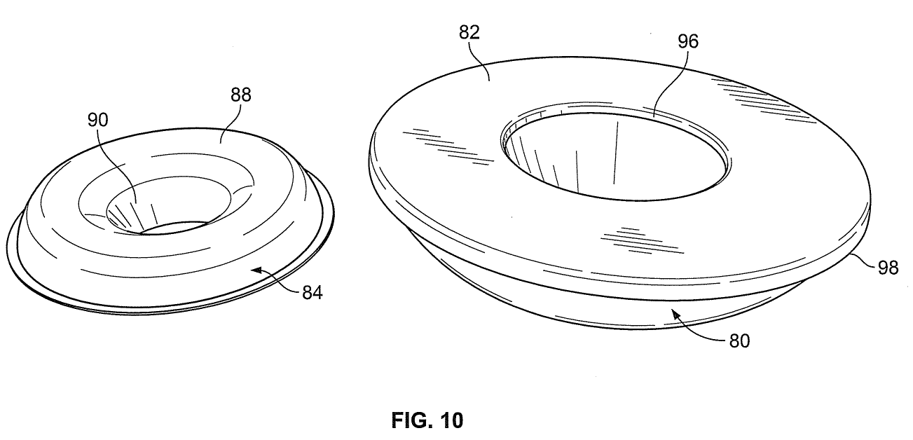

FIGS. 7 through 10 are of an embodiment adapted for use with a product sold by Medela, Inc. called the SoftShell nipple shield, used for protecting cracked or otherwise irritated nipples. Reference can also be made to U.S. Pat. No. 5,032,103. This embodiment has three parts: the nipple shield 80, which is a rigid shell; the flexible base 82 that mounts to the shell; and the new engorgement/edema adapter element 84.

The flexible base 82 is made of silicone material. It has an opening 96 and is provided with a lip 98 about its circumference for fitting over the edge of the shell 80.

The adapter element 84 is made of a silicone material having some rigidity. Adapter 84 has a toroidal-like ring part 86 with a contour (toward the breast in use) which yields the hummock-like raised portion 88 described in other versions above. The central region of the ring part 86 slopes to an opening 90, through which the nipple will extend in use.

The base 92 of the ring part 86 is made relatively planar, and there is a collar 94 surrounding the opening 90 and sized to fit within the opening 96 of the flexible base 82 normally used with the nipple shield. In this embodiment, three flanges 98 are arranged symmetrically around and extend outwardly from the exterior of the collar 94. These flanges engage the underside (inside) of the flexible base 82 to mount the engorgement/edema adapter 84 in place. The number of flanges 98 is not critical, provided that the flanges perform the required function. Furthermore, the opening 90 may be adjustable to accommodate different nipple sizes.

In use, the nipple shield 80 with adapter 84 is placed over the breast, and pressed against the breast for the desired massage. Note that another advantage of this embodiment is that a variety of adapters having differing materials, openings and/or contours can be provided. Alternatively, adapter 84 can be incorporated into flexible base 82. While the preferred material is flexible, a rigid base and/or adapter can be used.

FIGS. 12-13 depict another embodiment, the nipple shield 80 and the flexible base 82 being identical to those in the embodiment shown in FIGS. 7-10. The adapter 84′, however, has a larger diameter than that of the adapter 84, shown in FIGS. 7-10. The adapter 84′ includes a collar 94′ that is provided with a circumferential lip 98′, rather than a plurality of flanges 98. The lip 98′ engages the underside (inside) of opening 96 of the flexible base 82 to mount the engorgement/edema adapter 84′ in place. In use, the nipple shield 80 with adapter 84′ is placed over the breast, and pressed against the breast for the desired massage.

Although all the embodiments described herein have some flexible or resilient characteristic, it is entirely possible to make any of the treatment devices described herein of rigid material.

With the present invention, the pressure is applied to the surface of the device contacting the breast by a palm of a hand, a handle, a bra or similar garment, or via a breastshield of a breastpump. This results in the pressure applied to the breast being more uniformly distributed about the nipple of the breast, and not concentrated in a couple of locations, as results from the application of pressure from fingers located on opposite sides of the nipple. Further, the structure of the device of the present invention also facilitates the application of a more uniformly distributed pressure to the breast. In the embodiments discussed above, the surface of the device that contacts the breast is of such a construction (sufficient thickness and suitable material) that pressure applied in accordance with the present invention is applied over that entire surface to the breast.

While certain features and embodiments of the present application have been described in detail herein, it is to be understood that the application encompasses all modifications and enhancements within the scope and spirit of the following claims.

What is claimed is:

1. A device for application to a breast to treat engorgement, edema or similar conditions wherein undesired fluid has accumulated in the breast, comprising:
a member having a portion within which the breast nipple is received, said member having a force-applying surface about said portion which is adapted to engage with the breast in an area around the base of the nipple and extend a distance radially outwardly therefrom, and a part to which a motive force is applied to press said force-applying surface against the breast to press the breast along said distance so as to move fluid away from the nipple, the member having sufficient structure so that pressure can be applied over the entire force-applying surface to the breast.

2. A device for application to a breast to treat engorgement, edema or similar conditions wherein undesired fluid has accumulated in the breast, comprising:
an element having an orifice within which the nipple of a breast is received and a surface about said orifice,
said element being adapted to be hand-held so that said surface can be pressed onto the breast with the nipple in said orifice so as to apply a substantially uniformly distributed pressure about the nipple of the breast to move fluid away from the nipple.

3. The device of claim 2 wherein the element has a toroidal shape.

4. The device of claim 2 wherein the orifice is centrally located.

5. The device of claim 2 wherein the surface is on a proximal side of said device that engages the breast, and said proximal side is outwardly curved.

6. The device of claim 2 wherein said element is carried on a support structure.

7. The device of claim 6 wherein said support structure is a handle.

8. The device of claim 2 wherein said element is hollow.

9. A device for the treatment of excess fluid in the breast comprising:
a member having a portion within which the nipple of a breast is received and a surface about said portion that engages breast tissue surrounding said nipple,
said member being adapted to be placed within a bra or similar garment so that said surface can be pressed onto the breast with the nipple in said portion so as to move fluid away from the nipple.

10. The device of claim 9 wherein the member has a toroidal shape.

11. The device of claim 9 wherein the portion within which the nipple of the breast is received is an orifice that is centrally located.

12. The device of claim 9 wherein the surface is on a proximal side of said device that engages the breast, and said proximal side is outwardly curved.

13. A device for the treatment of excess fluid in the breast comprising:
a base; and
a dome-like element mounted to said base and having a well within which a nipple of a breast is received,
said dome-like element being adapted to be pressed against the breast with the nipple in said well so as to move fluid away from the nipple.

14. The device of claim 13 wherein said well curves outwardly into a raised area of said dome, said raised area surrounding said well.

15. The device of claim 13 wherein said dome-like element, when pressed against the breast with the nipple in said well, initially applies pressure primarily against the area immediately adjacent the nipple and then gradually expands said pressure radially outwardly as said dome-like element is pressed further against the breast.

16. A device for the treatment of engorgement or edema of the breast comprising:
a member having a portion within which the nipple of a breast is received and a surface about said portion,
said member being adapted to be mounted to a breastshield of a breastpump, so that said surface can be pressed onto the breast using the breastshield with the nipple in said portion so as to move fluid away from the nipple.

17. The device of claim 16 wherein said member comprises an insert adapted to fit within the breastshield.

18. The device of claim 17 wherein a portion of said insert has a toroidal shape.

19. The device of claim 16 wherein the portion within which the nipple of the breast is received is an orifice that is centrally located.

20. The device of claim 16 wherein said member is provided with a portion for fitting over a rim of the breastshield.

21. The device of claim 16 wherein said surface is outwardly curved relative to said breastshield.

22. A method of treating engorgement, edema or similar conditions wherein undesired fluid has accumulated in the breast, comprising the steps of:
providing a device comprising an element having an orifice within which the nipple of a breast is received and a surface about the orifice;
placing the nipple of the breast within the orifice of the element; and
pressing the surface onto the breast with the nipple in the orifice to move fluid away from the nipple without the use of fingers to provide a majority of the pressure applied to the surface.

23. The method of claim 22 further comprising the step of placing said device within a bra or similar garment, and using said bra or similar garment to press the surface onto the breast.

24. The method of claim 22 whereby said surface is pressed onto the breast manually.

25. The method of claim 22 further comprising the step of placing said device in a breastshield of a breastpump, and using said breastshield to press said device against the breast.

26. The method of claim 25 further including applying vacuum from the breastpump.

27. The method of claim 22 further including a handle to which said device is attached, and is used to press the surface onto the breast.

28. The method of claim 22 wherein the pressing step further comprises applying a substantially uniformly distributed pressure about the nipple of the breast.

29. A device for the treatment of the breast comprising:
a member having a portion within which a nipple of a breast is received and a surface about said portion and extending a distance therefrom but less than the entire breast, said member having a part adapted to press said surface of the member against the breast with the nipple in said portion so as to move fluid away from the nipple, the member having sufficient structure so that pressure can be applied over the entire surface to the breast.

30. The device of claim 29 wherein said member has a portion with a toroidal shape.

31. The device of claim 29 wherein the portion within which the nipple of the breast is received is an orifice that is centrally located.

32. The device of claim 29 wherein said surface is curved so as to have an apex that initially contacts the breast tissue and then moving said curved area then contacts said breast tissue as said device is pressed against the breast.

33. The device of claim 29 wherein said device is adapted to be mounted to a rigid nipple shield with said nipple shield then being used as said part adapted to press said surface of the member against said breast.

34. The device of claim 29 wherein said device is adapted to be supported on a breast handsfree.

35. The device of claim 34 wherein said device is adapted to be placed within a bra.

36. A device for treatment of engorgement or edema of the breast having a member for applying pressure against the breast so as to move fluid away from the nipple of the breast, the device adapted to be supported on the breast handsfree.
